(19) United States
(12) Patent Application Publication
(10) Pub. No.: US 2010/0189764 A1
(43) Pub. Date: Jul. 29, 2010

(54) BIOACTIVE MESH

(76) Inventors: Jonathan Thomas, New Haven, CT (US); Norman Aminuddin, Madison, CT (US); Joseph Hotter, Middletown, CT (US); Jon Reinprecht, Watertown, CT (US); Leslie Fronio, Annandale, NJ (US)

Correspondence Address: Tyco Healthcare Group LP, 60 MIDDLETOWN AVENUE, NORTH HAVEN, CT, 06473, US

(21) Appl. No.: 12/701,987

(22) Filed: Feb. 8, 2010

Related U.S. Application Data
(63) Continuation-in-part of application No. 11/886,934, filed on Jan. 5, 2009.
(60) Provisional application No. 60/664,134, filed on Mar. 22, 2005.

(51) Int. Cl. A61F 2/00; A61B 17/00; A61K 38/43; A61K 38/22; A61K 38/18; A61K 39/00; A61K 31/715; A61P 43/00

(52) U.S. Cl. 424/426; 606/151; 424/94.1; 514/12; 424/184.1; 514/54

(57) ABSTRACT

Surgical meshes include bicomponent fibers and/or microfibers. A bicomponent fiber or microfiber includes a non-absorbable inner core including at least one flexible polymer and an absorbable surface material including at least one rigid polymer. The absorbable surface material has a first stiffness and the non-absorbable inner core has a second stiffness which is lower than the first stiffness. The bicomponent fiber or microfiber surgical mesh is less flexible prior to implantation and more flexible following implantation in tissue. The mesh may be used for the treatment of hernias, vaginal prolapses, and other injuries.

### CROSS-REFERENCE TO RELATED APPLICATIONS

This application is a continuation-in-part of, and claims the benefit of and priority to, copending U.S. patent application Ser. No. 11/886,934, which is a National Phase of PCT/US2006/010330 filed Mar. 22, 2006, and also claims the benefit of and priority to U.S. Provisional Patent Application No. 60/664,134 filed Mar. 22, 2005, the entire disclosures of each of which are hereby incorporated by reference herein.

### BACKGROUND

1. Technical Field

The present disclosure relates to a coated surgical mesh that may be used in the treatment of hernias, uterovaginal prolapses and other related injuries.

2. Background of Related Art

A hernia is basically a defect resulting in the protrusion of part of an organ through the wall of a body cavity within which it is normally contained.

For example, a fairly common and well-known type of hernia is a defect in the lower abdominal wall resulting in a sac that may contain a portion of the intestine protruding through the abdominal wall. This is referred to as an inguinal hernia. A defect in the abdominal wall after surgery is referred to as an incisional hernia. Another type of hernia is a defect in the pelvic floor or other supporting structures resulting in a portion of the uterus, bladder, bowel or other surrounding tissue protruding through, e.g., the vaginal wall. This is usually referred to as a uterovaginal prolapse.

A common way of treating hernias is to repair the defect by sutures, whether or not the hernial sac is also sutured or repaired, in order that the protruding organ is contained in its normal position. As the defect generally comprises a weakening and attenuation leading to parting of tissues in a fascial wall, it is usually necessary to apply tension to the sutures in order to close the parted tissues. Thus, the fascial wall is generally pinched or tensioned around the area of the defect in order to close the parted tissues.

It has been suggested to make use of a surgical implant to overlay or close the weakened and parted tissues without the need to pinch or tension the surrounding tissue of the fascia. Such surgical implants generally comprise meshes and are now widely used in inguinal hernia repair. Meshes may be applied subcutaneously (i.e., under the skin) internally or externally of the abdominal wall and may be either absorbable or nonabsorbable depending on the nature and severity of the particular defect being treated. Meshes may be applied in combination with sutures to hold the mesh in place or, alternatively, with sutures that close the parted tissues as in a “non-mesh” technique.

Meshes are usually applied in open surgical procedures, although they may sometimes be applied in laparoscopic surgical procedures. An exemplary mesh for an inguinal hernia repair includes woven or knitted polypropylene, such as MARLEX® or PROLENE®. Such meshes have a number of desirable properties that make them effective for use in hernia repair. For example, they are made of materials that are suitably inert so as to be less likely to cause adverse reactions when implanted in the body. Furthermore, they are mechanically strong, cheap, easily sterilized, and easy to work with.

It has also been suggested to use meshes in the treatment of uterovaginal prolapse. Meshes that have been proposed for use in the repair of uterovaginal prolapse are similar to those that are used for the repair of inguinal hernias.

Conventionally, open procedures have been preferred for the treatment of hernias with meshes, as relatively broad access to the site of the defect is required to suitably implant and secure a mesh by sutures or such like. However, it is desirable to treat hernias, as when carrying out any surgery, with as little trauma to the patient as possible. Thus, the use of minimally invasive techniques has been suggested for the treatment of hernias. Such surgical techniques have not been considered to be useful in the treatment of uterovaginal prolapse with a mesh, as it has not been considered practical to position a mesh subcutaneously in the vaginal wall due to the difficulty in gaining direct access to this area.

In addition, one disadvantage of currently available meshes used in hernia repair is that they have jagged or rough edges. Thus, improvements to surgical implants such as meshes used to treat hernias and prolapses remain desirable.

### SUMMARY

The present disclosure provides medical devices made of bicomponent fibers and methods for making same. One may tailor the rigidity and stiffness of the fibers by selecting the types and amounts of materials utilized to form the core and the sheath of the fibers. In embodiments, a medical device of the present disclosure may include a mesh including at least one bicomponent fiber, the bicomponent fiber including a non-absorbable inner core including a first polymer, and a sheath surrounding at least a portion of the core including an absorbable rigid surface material including a second polymer, wherein the surgical mesh is more stiff prior to implantation and less stiff following implantation in tissue.

### BRIEF DESCRIPTION OF THE DRAWINGS

FIG. 1 is an illustration of a hernia.

FIG. 2 is an illustration of the hernia of FIG. 1 when intra-abdominal pressure is raised.

FIG. 3 is an illustration of the hernia of FIG. 1 after repair in accordance with the prior art.

FIG. 4 is an illustration of the hernia of FIG. 1 after an alternate repair in accordance with the prior art.

FIG. 5 is a schematic illustration of the female human vaginal area.

FIG. 6 is a cross-sectional view of the female human vaginal area along the line A-A of FIG. 5

FIGS. 7A and 7B illustrate meshes according to the present disclosure having a first shape.

FIG. 8A, 8B, 8C and 8D illustrate meshes according to the present disclosure having a second shape.

FIG. 9A, 9B, 9C and 9D illustrate meshes according to the present disclosure having a third shape.

FIG. 10A, 10B, 10C and 10D illustrate portions of meshes according to the present disclosure attached to a fastening device.

FIG. 11 depicts a perspective view of a fastener which may be used to attach a mesh of the present disclosure to tissue, illustrating a side view of a helical fastener.

FIG. 11A depicts another perspective view of a fastener which may be used to attach a mesh of the present disclosure to tissue, illustrating an end view of the helical fastener.

FIG. 11B depicts a schematic view of a fastener which may be used to attach a mesh of the present disclosure to tissue, illustrating a substantially collapsed helical fastener with a relatively small gap that has been partially inserted into tissue.

FIG. 11C depicts a schematic view of a fastener which may be used to attach a mesh of the present disclosure to tissue, illustrating the helical fastener depicted in FIG. 11B completely inserted into tissue.

FIG. 11D depicts a schematic view of a fastener which may be used to attach a mesh of the present disclosure to tissue, illustrating a substantially collapsed helical fastener with a relatively large gap that has been partially inserted into the tissue.

FIG. 11E depicts a schematic view of a fastener which may be used to attach a mesh of the present disclosure to tissue, illustrating the helical fastener depicted in FIG. 11D completely inserted into tissue.

FIG. 11F depicts a perspective view of another embodiment of a fastener which may be used to attach a mesh of the present disclosure to tissue, illustrating an end view of the helical fastener.

FIG. 12 depicts a perspective view of another embodiment of a fastener which may be used to attach a mesh of the present disclosure to tissue, illustrating a double helical fastener.

FIG. 12A is a front view of the double helical fastener of FIG. 12.

FIG. 12B is side view of the double helical fastener of FIG. 12.

FIG. 12C is a top view of the double helical fastener of FIG. 12.

FIG. 13 is a perspective view of yet another embodiment of a fastener which may be used to attach a mesh of the present disclosure to tissue, illustrating another design of a double helical fastener.

FIG. 13A is a front view of the double helical fastener of FIG. 13.

FIG. 13B is a side view of the double helical fastener of FIG. 13.

FIG. 13C is a top view of the double helical fastener of FIG. 13.

FIG. 14 is a perspective view of another fastener which may be used to attach a mesh of the present disclosure to tissue, illustrating a helical fastener with a central post.

FIG. 15 is a perspective view of an absorbable screw fastener which may be used to attach a mesh of the present disclosure to tissue.

FIG. 16 is another perspective view of the absorbable screw fastener of FIG. 15.

FIG. 17 is a longitudinal cross-sectional view of the absorbable screw fastener of FIG. 15 taken along line 3-3 of FIG. 15.

FIG. 18 is an orthogonal top view of the absorbable screw fastener of FIG. 17.

FIGS. 19A, 19B, and 19C depict a method of use of the bicomponent microfiber surgical mesh of the disclosure.

FIG. 20 is a partial cross-sectional perspective view of an embodiment of a surgical mesh in accordance with the present disclosure.

FIG. 21 is a cross-sectional view of an embodiment of a bicomponent fiber of a surgical mesh in accordance with the present disclosure.

FIG. 22 is a cross-sectional view of another embodiment of a bi-component fiber of a surgical mesh in accordance with the present disclosure.

FIG. 23 is a cross-sectional view of yet another embodiment of a bi-component fiber of a surgical mesh in accordance with the present disclosure.

FIG. 24 is a cross-sectional view of yet another embodiment of a bi-component fiber of a surgical mesh in accordance with the present disclosure.

### DETAILED DESCRIPTION

According to the present disclosure there is provided a surgical implant suitable for treatment of a hernia, prolapse, or other similar injury. The implant includes a mesh having a maximum residual mass density of 50 g/m² or less. The residual mass density is the mass density of the mesh after implantation and the absorption of any bioabsorbable coatings. In one embodiment the maximum residual mass density may be less than 30 g/m², while in another useful embodiment the maximum residual mass density may be less than 25 g/m². Thus, in embodiments the maximum residual mass density may be from about 5 g/m² to about 50 g/m², in embodiments from about 15 g/m² to about 40 g/m², in embodiments from about 25 g/m² to about 35 g/m².

The mesh of the present disclosure is made of strands which, in turn, may be made of any suitable biocompatible material. Suitable materials from which the mesh can be made should have the following characteristics: sufficient tensile strength to support a fascial wall during repair of a defect in the fascial wall causing a hernia; sufficiently inert to avoid foreign body reactions when retained in the human body for long periods of time; easily sterilized to prevent the introduction of infection when the mesh is implanted in the human body; and have suitably easy handling characteristics for placement in the desired location in the body. The mesh should be sufficiently pliable to conform to a fascial wall and flex with movement of the wall, while being sufficiently rigid to retain its mesh shape following transport through a laparoscopic device.

The mesh includes filaments, major spaces, and pores. The filaments of the mesh may be formed by at least two strands, the major spaces formed between the filaments providing the surgical implant with the necessary strength, the filaments arranged such that pores are formed in the strands themselves. Alternatively the filaments may be formed by monofilaments that are arranged to form loops that give rise to the pores in the strands. The filaments may be spaced apart to form major spaces of from about 0.5 mm to about 10 mm between the filaments. In one useful embodiment the filaments may be spaced apart to form major spaces of from about 2 mm to about 8 mm between the filaments. The use of mesh having filaments spaced apart in accordance with the present disclosure has the advantage of reducing the foreign body mass that is implanted in the human body, while maintaining sufficient tensile strength to securely support the defect and tissue being repaired by the mesh.

The strands of the mesh may have a diameter of less than about 600 μm, in embodiments from about 25 μm to about 600 μm, in embodiments from about 50 μm to about 200 μm. In other embodiments, a multifilament mesh may include several filaments which, when combined or bundled (e.g., braided, woven, knit, and the like), may total in diameter to less than about 600 μm, in embodiments from about 25 μm to about 600 μm, in embodiments from about 50 μm to about 200 μm.

The strands and filaments may be braided, twisted, aligned, or otherwise joined to foam a variety of different mesh shapes. In embodiments, at least two strands or filaments may form a yarn for use in forming the mesh. In other embodiments, multiple strands or filaments may be braided, twisted, aligned, or otherwise joined to form a multifilament yarn. The mesh may be assembled from a plurality of filaments or yarns. The fibers/yarns may be woven, knitted, interlaced, braided, or formed into a surgical mesh by non-woven techniques. The structure of the mesh will vary depending upon the assembling technique utilized to form the mesh, as well as other factors, such as the type of fibers used, the tension at which the fibers are held, and the mechanical properties required of the mesh.

In embodiments, knitting may be utilized to form a mesh of the present disclosure. Knitting involves, in embodiments, the intermeshing of fibers or yarns to form loops or inter-looping of the fibers or yarns. In some embodiments, fibers and/or yarns may be warp-knitted thereby creating vertical interlocking loop chains and/or may be weft-knitted thereby creating rows of interlocking loop stitches across the mesh. In other embodiments, weaving may be utilized to a mesh of the present disclosure. Weaving may include, in embodiments, the intersection of two sets of straight yarns, warp and weft, which cross and interweave at right angles to each other, or the interlacing of two yarns at right angles to each other. In some embodiments the strands may be arranged to form a net mesh which has isotropic or near isotropic tensile strength and elasticity.

In embodiments, the fibers/yarns may be nonwoven and formed by mechanically, chemically, or thermally bonding the fibers/yarns into a sheet or web. For example, fibers/yarns may be mechanically bound by entangling the fibers/yarns to form the mesh by means other than knitting or weaving, such as matting, pressing, stitch-bonding, needlepunching, or otherwise interlocking the fibers/yarns to form a binderless network of fibers/yarns. In other embodiments, the fibers/yarns of the mesh may be chemically bound by use of an adhesive, such as a hot melt adhesive, or thermally bound by applying a binder, such as a powder, paste, or melt, and melting the binder on the sheet or web of fibers/yarns.

As the surgical implant includes narrow strands that are spaced by relatively wide gaps, tissue may be slow to grow into the mesh of the present disclosure. Thus, it may be desirable for the mesh to have means for promoting tissue ingrowth. In embodiments, it may be desirable to provide pores in the strands of the mesh to aid tissue ingrowth and to provide a surface to which tissue may more easily adhere.

As described above, at least one filament may be interwoven or knitted to produce strands having pores which, in turn, are utilized to faun a mesh of the present disclosure. For manufacturing reasons, it may be desirable to use two filaments to form pores in the strands of the mesh to assist tissue ingrowth. However, if one filament can be suitably knotted or twisted to form pores of suitable dimensions, this single filament may be used to form the strands of the mesh.

The pores of the mesh of the present disclosure may be of a size that permit fibroblast through-growth and ordered collagen laydown, resulting in integration of the mesh into the body. For example, the woven/knitted filaments create pores in the strands that are from about 50 μm to about 200 μm in diameter, in embodiments from about 55 μm to about 75 μm in diameter. Alternatively, rings or loops of material of from about 50 μm to about 200 μm in diameter may be adhered to or formed on the strands of the mesh to provide additional pores on the strands.

Due to the wide spacing between strands of the mesh of the present disclosure and the small diameter of the filaments, problems found with currently available meshes, i.e., their jagged and/or rough edges, are mitigated.

Due to the variety of sizes of such defects, and of the various fascia that may need repair by the implant, the implant may be of any suitable size. In one embodiment, the surgical implant is of a width from about 1 cm to about 10 cm and a length from about 1 cm to about 10 cm.

In some embodiments the filaments may be made of a plastic or similar synthetic non-absorbable material. Suitable materials include, polyolefins such as polyethylene and polypropylene including atactic, isotactic, syndiotactic, and blends thereof; ultra high molecular weight polyethylene; polyethylene glycols; polyethylene oxides; polyisobutylene and ethylene-alpha olefin copolymers; fluorinated polyolefins such as fluoroethylenes, fluoropropylenes, fluoroPEGSs, and polytetrafluoroethylene; polyamides such as nylon, Nylon 6, Nylon 6,6, Nylon 6,10, Nylon 11, Nylon 12, and polycaprolactam; polyamines; polyimines; polyesters such as polyethylene terephthalate, polyethylene naphthalate, polytrimethylene terephthalate, and polybutylene terephthalate; polyethers; polybutester; polytetramethylene ether glycol; 1,4-butanediol; polyurethanes; acrylic polymers; methacrylics; vinyl halide polymers such as polyvinyl chloride; polyvinyl alcohols; polyvinyl ethers such as polyvinyl methyl ether; polyvinylidene halides such as polyvinylidene fluoride and polyvinylidene chloride; polychlorofluoroethylene; polyacrylonitrile; polyaryletherketones; polyvinyl ketones; polyvinyl aromatics such as polystyrene; polyvinyl esters such as polyvinyl acetate; etheylene-methyl methacrylate copolymers; acrylonitrile-styrene copolymers; ABS resins; ethylene-vinyl acetate copolymers; alkyd resins; polycarbonates; polyoxymethylenes; polyphosphazines; polyimides; epoxy resins; aramids; rayon; rayon-triacetate; spandex; silicones; and copolymers and combinations thereof. Additionally, non-biodegradable polymers and monomers may be combined with each other to create a core of a fiber, for example a fiber possessing a core-sheath configuration, sometimes referred to herein as a bicomponent fiber.

In another embodiment the filaments of the mesh may be made of an absorbable material such as an aliphatic polyester. Absorbable materials are absorbed by biological tissues and disappear in vivo at the end of a given period, which can vary, for example, from hours to several months, depending on the chemical nature of the material. The biocompatible material may provide rigidity to the filaments and any device, in embodiments a mesh, produced therefrom.

Absorbable materials include both natural and synthetic biodegradable polymers and copolymers. Representative natural biodegradable polymers include: polysaccharides such as alginate, dextran, chitin, chitosan, hyaluronic acid, cellulose, collagen, gelatin, fucans, glycosaminoglycans, and chemical derivatives thereof (substitutions and/or additions of chemical groups include, for example, alkyl, alkylene, amine, sulfate, hydroxylations, carboxylations, oxidations, and other modifications routinely made by those skilled in the art); catgut; silk; linen; cotton; and proteins such as albumin, casein, zein, silk, soybean protein, and copolymers and blends thereof; alone or in combination with synthetic polymers.

Synthetically modified natural polymers which may be used to form filaments include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, and cellulose sulfate sodium salt. These are collectively referred to herein as “celluloses.”

Representative synthetic degradable polymers which may be utilized to form filaments include polyhydroxy acids prepared from lactone monomers such as glycolide, lactide, caprolactone, ε-caprolactone, valerolactone, and δ-valerolactone, carbonates (e.g., trimethylene carbonate, tetramethylene carbonate, and the like), dioxanones (e.g., 1,4-dioxanone and p-dioxanone), 1,dioxepanones (e.g., 1,4-dioxepan-2-one and 1,5-dioxepan-2-one), and combinations thereof. Polymers formed therefrom include: polylactides; poly(lactic acid); polyglycolides; poly(glycolic acid); poly(trimethylene carbonate); poly(dioxanone); poly(hydroxybutyric acid); poly(hydroxyvaleric acid); poly(lactide-co-(ε-caprolactone-)); poly(glycolide-co-(ε-caprolactone)); polycarbonates; poly(pseudo amino acids); poly(amino acids); poly(hydroxyalkanoate)s such as polyhydroxybutyrate, polyhydroxyvalerate, poly(3-hydroxybutyrate-co-3-hydroxyvalerate), polyhydroxyoctanoate, and polyhydroxyhexanoate; polyalkylene oxalates; polyoxaesters; polyanhydrides; polyester anyhydrides; polyortho esters; polyphosphazenes; and copolymers, block copolymers, homopolymers, blends, and combinations thereof.

Synthetic degradable polymers also include hydrophilic vinyl polymers expanded to include phosphoryl choline such as 2-methacryloyloxyethyl phosphorylcholine, hydroxamates, vinyl furanones and their copolymers, and quaternary ammonia; as well as various alkylene oxide copolymers in combination with other polymers such as lactones, orthoesters, and hydroxybutyrates, for example.

Rapidly bioerodible polymers, such as poly(lactide-co-glycolide)s, polyanhydrides, and polyorthoesters, which have carboxylic groups exposed on the external surface as the smooth surface of the polymer erodes, may also be used.

Other degradable polymers include polyphosphazenes; polypropylene fumarates; polyimides; polymer drugs such as polyamines; perfluoroalkoxy polymers; fluorinated ethylene/propylene copolymers; PEG-lactone copolymers; PEG-polyorthoester copolymers; blends and combinations thereof.

It can be appreciated that filaments which are made in part of absorbable material would allow better surgical handling and enable the implant to have minimal mass following implantation in the body.

In yet another embodiment, filaments of the mesh may be made of a material that has memory. Shape memory polymers are generally characterized as phase segregated linear block co-polymers having a hard segment and a soft segment. The hard segment is typically crystalline, with a defined melting point, and the soft segment is typically amorphous, with a defined glass transition temperature. In embodiments, however, the hard segment may be amorphous and have a glass transition temperature and the soft segment may be crystalline and have a melting point. The melting point or glass transition temperature of the soft segment is substantially less than the melting point or glass transition temperature of the hard segment.

Suitable monomers and polymers used to prepare hard and soft segments of shape memory polymers include caprolactone, dioxanone, lactide, glycolide, polyacrylates, polyamides, polysiloxanes, polyurethanes, polyether amides, polyurethane/ureas, polyether esters, urethane/butadiene copolymers, and combinations thereof.

When the shape memory polymer is heated above the melting point of the hard segment, the material can be shaped. This shape can be memorized by cooling the shape memory polymer below the melting point of the hard segment. When the shape memory polymer is cooled below the glass transition temperature of the soft segment while the shape is deformed, a new temporary shape can be set. The original shape can be recovered by heating the material above the glass transition temperature of the soft segment but below the melting point of the hard segment. The recovery of the original shape, which is induced by an increase in temperature, is called the thermal shape memory effect.

A mesh with memory urges the surgical implant to adopt a flat conformation. Such an implant may have a curved perimeter, i.e., few or no corners or apexes, as sharp corners increase the likelihood of edge erosion and infection. The specific shape will, however, vary according to the intended use of the implant.

In embodiments of the present disclosure, filaments may be formed from polypropylene having a diameter of from about 0.07 mm to about 0.08 mm, wherein the strands making up the mesh are spaced to form spaces in the mesh of from about 2 mm to about 5 mm.

In other embodiments, filaments may be formed from polyester, such as PET, having a diameter of from about 0.05 mm to about 0.2 mm, wherein the strands are spaced to form spaces in the mesh of from about 0.75 mm to about 5 mm.

As noted above, filaments used to produce the strands of the mesh of the present disclosure may be made of bicomponent fibers or microfibers. Bicomponent fibers may include a core material and a surface material. The core of the bicomponent fiber may be formed of any suitable biocompatible material, for example, a polymer or similar synthetic non-absorbable material as described above. The surface of the bicomponent fiber may include an absorbable surface material. The absorbable surface material may be made of any suitable absorbable biocompatible material as described above.

In embodiments, the bicomponent fibers may include a nonabsorbable or long lasting absorbable core and a shorter lasting absorbable surface material. The absorbable surface material provides stiffness and rigidity to the mesh for ease of implantation and degrades to leave a pliant, flexible core for long-term implantation in the body. The surface material of the bicomponent fiber may be absorbed by the body within a number of hours, such that only the core portion is left in the body for an extended period of time, typically for a long enough period of time to enable tissue ingrowth. In other embodiments, the absorbable portion may be absorbed from about 2 days to about 180 days post-implantation. In yet other embodiments, the absorbable portion may be a rapidly bioerodible polymer or other fast absorbing polymer, as described above, having a fast degradation rate of just a few hours to less than an hour.

In certain embodiments, the sheath component is more stiff than the core component. Once implanted, degradation of the stiffer sheath may, in general, lead to a more flexible mesh, exposing and leaving a less stiff core. In embodiments, the glass transition temperature of the sheath may determine stiffness values. For example, at certain temperatures, the material properties, i.e., stiffness, of the sheath may change, rendering a less stiff, more flexible and pliable material.

One skilled in the art can alter the degradation mechanism of the sheath by changing parameters including, but not limited to, polymer composition and chemistry, density, morphology, crystal structure, solubility, thermal properties, molecular weight, size, porosity and pore size, wettability, and processing parameters. It is within the purview of one skilled in the art to alter the processing of the implant to control the various parameters listed above including, but not limited to, polymer crystallinity and morphology, density, molecular weight, porosity and pore size.

Alternatively, the core of the bicomponent fiber or microfiber may be less stiff as compared to the sheath. For example, the core material may exhibit a stiffness, as determined by Gurley stiffness testing. Such testing methods are within the purview of those skilled in the art and include those disclosed, for example, in U.S. Pat. No. 3,630,205, the entire disclosure of which is incorporated by reference herein.

In embodiments, Gurley stiffness may be measured with a motor-operated Gurley Stiffness Tester (Model 4171E) manufactured by Gurley Precision Instruments (Troy, New York) following the manufacturer's directions.

In embodiments, a suitable instrument for determining Gurley stiffness includes a balanced pendulum or pointer, which is center pivoted and which can be variously weighted below its center with a removable weight. The pointer moves parallel to a “sine” scale graduated in both directions. In the test, monofilament strands are inserted in a jig constructed with 10 or 20 parallel holes drilled on ⅛-inch centers. The strands are inserted so that at least ¼ inch protrudes out of the holes. A razor blade may then be used to closely shave the strand tips which extend past the jig guide. The exposed ¼-inch of the strands are then bound between ½-inch masking tape and the width of the sample is trimmed to one inch. The total length of each sample is one inch with ¼-inch of the total length bound in masking tape.

The tape bound end of the sample is clamped on the motor-driven arm of the Gurley instrument so that the clamp-bending bar lies one-half inch above the edge of the swinging pendulum. This gives the sample strands ¼-inch of extension beyond the tip of the swinging pendulum.

When the motor-driven arm presses the monofilaments against the edge of the pendulum, the pointer is deflected until the sample scrapes past the pendulum and may be read on the scale. The resistance of the pendulum and thus the sensitivity of the machine to materials of different stiffness can be adjusted in two ways: by changing the distance from the fulcrum of the weight and by changing the weight itself.

The machine is operated for one or two cycles to adjust the weight-distance combination if necessary. This adjustment should be made so that the average reading will fall between 1.0 and 7.0 Gurley units. (A cycle is defined as a left plus a right swing of the pointer. A Gurley unit is the unit reading marked on the sine scale.) After the necessary adjustments are made, the machine is operated for 10 cycles recording the average of the two (left and right) values for each cycle. After each half cycle, the oscillation of the pendulum is stopped before continuing. The stiffness of the sample may be calculated by use of the following formula:

$$GurleyStiffness ( mg ) ≡ 1000 \frac{{RDML}_{R}^{2}}{10 {VW}_{R}}$$

R is the Gurley scale reading
D is the distance from weight to pivot, mm
V is the distance from tip of swinging pendulum to pivot=127.0 mm
M is the weight, g
L_{R} is the Length ratio=Lₜₑₛₜ/76.2 mm
W_{R} is the width ratio=Wₜₑₛₜ/25.4 mm

It should be understood that the above stiffness value may change as a function of fiber diameter. Exemplary materials that may be used and their stiffnesses are set forth below in Table 1. For example, in looking at Table 1, PLA (poly lactic acid) monofilament has a higher average stiffness compared to PP (polypropylene), PET (polyethylene terephthalate), and Surgipro™ and would therefor make a suitable coating for those filaments.

TABLE 1

| Gurley Stiffness Testing | | | | | | |
|---|---|---|---|---|---|---|
| | | Avg. | | | | |
| | | Stiff- | Machine Settings | | | |
| | | ness | | | Weight | |
| Material | Diameter | (mg) | Length | Width | Position | Weight |
| Surgipro ™ | 0.148 mm | 28.670 | 1″ | 1″ | 2″ | 5 g |
| II 5/0 | | | | | | |
| PLA | 0.150 mm | 53.378 | 1″ | 1″ | 4″ | 5 g |
| Mono- | | | | | | |
| filament | | | | | | |
| Maxon ™ | 0.115 mm | 6.818 | 1″ | 1″ | 1″ | 5 g |
| 6/0 | | | | | | |
| PP | 0.100 mm | 8.380 | 1″ | 1″ | 1″ | 5 g |
| Mono- | | | | | | |
| filament | | | | | | |
| PET | 0.08 mm | 7.981 | 1″ | 1″ | 1″ | 5 g |
| Mono- | | | | | | |
| filament | | | | | | |

Following implantation, the core may be capable of forming to, and flexing with, the fascia to which it is attached.

In embodiments, the bicomponent fibers are microfibers having a diameter of from about 40 microns to about 300 microns. In some embodiments, the core of the bicomponent fiber may have a diameter of from about 59.7 microns to about 156.1 microns at least for monofilaments sizes 4/0, 5/0, and 6/0. The non-absorbable inner core may be present in an amount, for example, from about 50 to about 80 percent by area/volume.

In embodiments, the absorbable surface material of the bicomponent fibers is thin for rapid degradation, while still providing the desired rigidity. Thus, a large majority of the cross-section of the fiber is the core as illustrated in FIGS. 21-23. The core may be from about 50% to about 95% of the cross-sectional area of the fiber and the absorbable surface material is from about 5% to about 50% of the cross-sectional area (assuming equal density of the core and sheath). In embodiments, the core is from about 50% to about 80% of the cross-sectional area of the fiber, while the absorbable surface material is from about 20% to about 50% of the cross-sectional area of the fiber. In some embodiments, the absorbable surface material has a thickness from about 4.5 microns to about 25.6 microns, at least for a monofilament size 4/0, 5/0, and 6/0, assuming concentric and single islet-in-the-sea configuration. Suitable examples are listed in Table 2 below.

TABLE 2

| | USP Size | | |
|---|---|---|---|
| | 4/0 | 5/0 | 6/0 |
| Diameter Range (micron) | 150-199 | 100-149 | 70-99 |
| Average Diameter (micron) | 174.5 | 124.5 | 84.5 |
| 20/80 Core Diameter (micron) | 156.1 | 111.3 | 75.5 |
| 20/80 Outer Layer Thickness (micron) | 9.2 | 6.6 | 4.5 |
| 50/50 Core Diameter (micron) | 123.3 | 88.1 | 59.7 |
| 50/50 Outer Layer Thickness (micron) | 25.6 | 18.2 | 12.4 |

The absorbable surface material may be applied to the core of the bicomponent fiber by any means within the purview of those skilled in the art including: extrusion; co-extrusion; pultrusion; gel spinning with one of the aforementioned processes; melt coating; spray coating; ultrasonic spray coating; electrostatic coating; powder coating; solvent/immersion coating such as dipping; spraying; solvent evaporation; sheath heat crimping; chemical surface modification; combinations thereof, and the like. In embodiments, powder coating may be accomplished by coating a dry form of absorbable polymer particles and melting them to form a conformal coating. In other embodiments, the absorbable surface material may be applied to the core by chemical surface modification which may include energy based polymerization, such as ultraviolet (UV), gamma, heat and/or chemical initiated processes, or by e-beam polymerization which may include polymerizing degradable polymers in the presence of the yarn/fiber utilized to form the core.

In embodiments, the surface of the core fiber may include some degree of porosity to help anchor or impregnate at least a portion of the outer layer into the core. The porosity of the core fiber may also provide for tissue ingrowth following degradation/absorption of the outer layer. In embodiments, porosity is achieved by roughening the surface of the core fiber.

Where applied as a coating, the surface material may be in a solution including a biocompatible solvent such as hexafluoroisopropanol, acetone, isopropanol, methylene chloride, chloroform, combinations thereof, and the like.

In embodiments, the surface material may form a sheath surrounding or encompassing at least a portion of the core.

The absorbable surface material of the bicomponent fiber or microfiber may be rigid or stiff as compared to the core. In some embodiments, the sheath exhibits a glass transition temperature (Tg) which is less than body temperature (37° C.) but greater than room temperature (25° C.), such that upon implantation and exposure to a temperature higher than the Tg temperature, the polymer sheath transitions to a less stiff (more rubbery) state, compared to the stiffness at room temperature. In other embodiments, the absorbable surface material exhibits a stiffness at temperatures from about 25° C. to about 37° C., thereby providing rigidity to the mesh which will be easier to manipulate and implant during a laparoscopic procedure. At a later time point, once the sheath begins degradation, the mesh may become less stiff and more pliable. The absorbable surface material may exhibit stiffness, as determined by a modified Gurley stiffness testing as described above for the core, in the amounts as set forth above in Table 1.

In embodiments, the absorbable surface material of the bicomponent fiber provides stiffness to the entire bicomponent fiber and thereby provides stiffness to the mesh as a whole. In other embodiments, the absorbable surface material exhibits stiffness such that, when the surgical mesh is folded, the mesh may spontaneously unfold to its original geometric shape. In yet another embodiment, the absorbable surface material of the bicomponent fiber allows the surgical mesh to reform into its original geometric shape following transport through a laparoscopic device into a body cavity.

In embodiments, the bicomponent fibers are constructed, at least in part, using shape memory polymers. In embodiments, the absorbable surface may include a shape memory polymer having a flat, original shape which is cooled to a rolled-up configuration for insertion through a laparoscopic port. The flat, original shape is recovered after implantation as the mesh is heated to the temperature of the body, thus flattening out for placement against the abdominal wall or other desired location.

The absorbable surface material of the bicomponent fibers provides the surgical mesh with characteristics required for surgical handling. After insertion in the body, the absorbable surface material of the bicomponent fiber is absorbed by the body leaving behind the reduced mass of the core material as the mesh.

In embodiments, the absorbable surface material of the bicomponent fibers may be absorbed by the body within a number of seconds, minutes, hours, or days, such that only the core remains in the body. In embodiments the absorbable surface material may be absorbed within a period of time from about 2 days to about 180 days.

When the absorbable surface material and the core are combined, they form a monofilament bicomponent fiber. In embodiments, monofilaments used to produce the mesh implant may have a diameter from about 70 microns to about 199 microns, depending on the size of the filaments utilized. Other sizes outside of this range are also envisioned.

The mesh of the present disclosure may possess a bioactive agent, which may be in the form of a coating. The term “bioactive agent”, as used herein, is used in its broadest sense and includes any substance or mixture of substances that have clinical use. Consequently, bioactive agents may or may not have pharmacological activity per se, e.g., a dye. Alternatively, a bioactive agent could be any agent which provides a therapeutic or prophylactic effect; a compound that affects or participates in tissue growth, cell growth and/or cell differentiation; a compound that may be able to invoke or prevent a biological action such as an immune response; or a compound that could play any other role in one or more biological processes. Moreover, any agent which may enhance tissue repair, limit the risk of sepsis, and modulate the mechanical properties of the mesh (e.g., the swelling rate in water, tensile strength, etc.) may be added during the preparation of the mesh or may be coated on or into the major spaces or pores of the mesh.

Examples of classes of bioactive agents which may be utilized in accordance with the present disclosure include antimicrobials, analgesics, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, gastrointestinal drugs, diuretics, steroids, lipids, lipopolysaccharides, polysaccharides, and enzymes. It is also intended that combinations of bioactive agents may be used.

Other bioactive agents which may be in the present disclosure include: local anesthetics; non-steroidal antifertility agents; parasympathomimetic agents; psychotherapeutic agents; tranquilizers; decongestants; sedative hypnotics; steroids; sulfonamides; sympathomimetic agents; vaccines; vitamins; antimalarials; anti-migraine agents; anti-parkinson agents such as L-dopa; anti-spasmodics; anticholinergic agents (e.g., oxybutynin); antitussives; bronchodilators; cardiovascular agents such as coronary vasodilators and nitroglycerin; alkaloids; analgesics; narcotics such as codeine, dihydrocodeinone, meperidine, morphine and the like; non-narcotics such as salicylates, aspirin, acetaminophen, d-propoxyphene and the like; opioid receptor antagonists such as naltrexone and naloxone; anti-cancer agents; anti-convulsants; anti-emetics; antihistamines; anti-inflammatory agents such as hormonal agents, hydrocortisone, prednisolone, prednisone, non-hormonal agents, allopurinol, indomethacin, phenylbutazone and the like; prostaglandins and cytotoxic drugs; estrogens; antibacterials; antibiotics; anti-fungals; anti-virals; anticoagulants; anticonvulsants; antidepressants; antihistamines; and immunological agents.

Other examples of suitable bioactive agents which may be included in the present disclosure include: viruses and cells; peptides, polypeptides and proteins, as well as analogs, muteins, and active fragments thereof; immunoglobulins; antibodies; cytokines (e.g., lymphokines, monokines, chemokines); blood clotting factors; hemopoietic factors; interleukins (IL-2, IL-3, IL-4, IL-6); interferons (β-IFN, (α-IFN and γ-IFN)); erythropoietin; nucleases; tumor necrosis factor; colony stimulating factors (e.g., GCSF, GM-CSF, MCSF); insulin; anti-tumor agents and tumor suppressors; blood proteins; gonadotropins (e.g., FSH, LH, CG, etc.); hormones and hormone analogs (e.g., growth hormone); vaccines (e.g., tumoral, bacterial and viral antigens); somatostatin; antigens; blood coagulation factors; growth factors (e.g., nerve growth factor, insulin-like growth factor); protein inhibitors; protein antagonists; protein agonists; nucleic acids such as antisense molecules, DNA, and RNA; oligonucleotides; and ribozymes.

A single bioactive agent may be utilized or, in alternate embodiments, any combination of bioactive agents may be utilized.

In some embodiments, a bioactive coating may be applied to the mesh as a composition or coating containing one or more bioactive agents, or bioactive agent(s) dispersed in a suitable biocompatible solvent. Suitable solvents for particular bioactive agents are within the purview of those skilled in the art.

In embodiments, bicomponent fibers may be used for both forming a device of the present disclosure as well as delivery of a bioactive agent. Thus, in some embodiments, at least one bioactive agent may be combined or separately applied to the individual bicomponent fibers of the mesh as a coating for quick release of the bioactive agent, to the surface material of the bi-component fibers for release during degradation of the absorbable surface material, and/or to the core for long-term release over time. For example, the bioactive agents may be freely admixed with the polymers for forming the core or sheath of the bicomponent fiber or may be tethered to the polymers through any suitable chemical bonds.

In other embodiments, the bioactive coating may be combined with a bioabsorbable material. Suitable bioabsorbable materials include those listed above. The bioactive coating may have any thickness or bulk and may be utilized to provide the mesh with suitable handling characteristics.

Any coating composition containing the bioactive agent may encapsulate an entire filament, strand or mesh. Alternatively, the bioactive coating may be applied to one or more sides of a filament, strand or mesh. Such a coating will improve the desired therapeutic characteristics of the mesh.

The bioactive agent may be applied to the mesh utilizing any suitable method known to those skilled in the art. Some examples include, but are not limited to, spraying, dipping, layering, calendaring, etc. The bioactive agent may also be otherwise incorporated or applied to the mesh.

Where the bioactive coating includes an absorbable material, the coating may be released into the body within a period of time from about 1 hour to about 21 days following implantation. In one embodiment the coating may be released from about 2 days to about 14 days following implantation. In another useful embodiment, the coating may be released from about 2 hours to about 14 days following implantation.

The rate of release of a bioactive agent from the bioactive coating on a mesh of the present disclosure can be controlled by any means within the purview of one skilled in the art. Some examples include, but are not limited to, the depth of the bioactive agent from the surface of the coating; the size of the bioactive agent; the hydrophilicty of the bioactive agent; and the strength of physical and physical-chemical interaction between the bioactive agent, the bioactive coating and/or the mesh material. By properly controlling some of these factors, a controlled release of a bioactive agent from the mesh of the present disclosure can be achieved.

In another embodiment, the mesh of the present disclosure may comprise a backing strip which may be releasably attached to the mesh. The backing strip may be formed from a range of materials, including plastics, and may be releasably attached by an adhesive.

The releasable attachment of a backing strip to the mesh may provide a more substantial and less flexible surgical implant, which may be more easily handled by a surgeon. Following suitable placement of the surgical implant, the backing strip can be removed from the surgical implant, the surgical implant being retained in the body and the backing material being removed by the surgeon. The surgical implant can therefore benefit from reduced mass while still providing characteristics required for surgical handling.

A surgical mesh formed from the bicomponent fibers and/or microfibers may be applied during open surgery. During open surgery, the rigidity of the surgical mesh will allow for ease of handling by the surgeon. Following application and attachment of the mesh, the absorbable surface material may dissolve leaving behind a sufficiently strong mesh needed to maintain the long term integrity of the hernia repair. The remaining mesh will be flexible, forming to the abdominal wall. The mesh may also be used, in embodiments, to prevent and/or reduce adhesions which may otherwise occur between a mesh and tissue.

Alternatively, the surgical mesh may be applied during minimally invasive surgery. Laparoscopic surgical procedures are minimally invasive procedures in which operations are carried out within the body by using elongated deployment devices, inserted through small entrance openings in the body. The initial opening in the body tissue to allow passage of the endoscopic or laparoscopic devices to the interior of the body may be a natural passageway of the body, or it can be created by a tissue piercing device such as a trocar. During laparoscopic procedures, narrow punctures or incisions may be made, thereby minimizing trauma to the body cavity and reducing patient recovery time.

Laparoscopic deployment devices may be used for transferring a mesh into a body cavity. Such devices are within the purview of those skilled in the art and include, for example, the devices disclosed in U.S. Patent Application Publication Nos. 2006/0229640, 2006/0200170, and/or 2006/0200169, the entire disclosures of each of which are incorporated herein by reference.

A mesh according to the present disclosure can be inserted through a small incision (e.g., from about 1 cm to about 2 cm in length) with the use of a laparoscopic deployment device, trocar, or other device. The mesh may be rolled or folded so as to fit within the device for transfer into the body cavity. Upon exiting the transfer device, the absorbable surface material of the bicomponent microfiber provides sufficient stiffness to reopen the rolled or folded mesh into its original geometric shape.

In embodiments, the surface material of the bicomponent fibers or microfibers provides the surgical implant with characteristics required for surgical handling. After insertion in the body, the surface material of the bicomponent fiber may be absorbed by the body leaving behind the reduced mass of the core material as the strands of the mesh.

In another embodiment, the bicomponent fibers or microfibers may be made of a core material which may be rapidly absorbed by the body and a surface material which is not absorbed as rapidly, i.e., it is absorbed over a longer period of time than the core.

It may be desirable to provide a variety of implants having different sizes so that a surgeon can select an implant of suitable size to treat a particular patient. This allows implants to be completely formed before delivery, ensuring that the smooth edge of the implant is properly formed under the control of the manufacturer. The surgeon would thus have a variety of differently sized (and/or shaped) implants to select the appropriate implant to use after assessment of the patient.

In another embodiment the mesh can be cut to any desired size. The cutting may be carried out by a surgeon or nurse under sterile conditions such that the surgeon need not have many differently sized implants on hand, but can simply cut a mesh to the desired size of the implant after assessment of the patient. In other words, the implant may be supplied in a large size and be capable of being cut to a smaller size, as desired.

Even where the cutting of the mesh causes an unfinished edge of the mesh to be produced, this unfinished mesh is not likely to cause the same problems as the rough and jagged edges of the implants of the prior art, due to the fewer strands, smaller diameter filaments and treatment of the mesh with a coating which protects the tissue from the mesh during the surgical procedure when damage is most likely to occur.

Different shapes are suitable for repairing different defects in fascial tissue, and thus by providing a surgical implant which can be cut to a range of shapes, a wide range of defects in fascial tissue can be treated.

More broadly, the present disclosure recognizes that the implant can have any shape that conforms with an anatomical surface of a human or animal body that may be subject to a defect to be repaired by the implant.

Typically an anterior uterovaginal prolapse is elliptical in shape or a truncated ellipse, whereas a posterior prolapse is circular or ovoid in shape. Accordingly, the implant shape may be any one of elliptical or truncated ellipse, round, circular, oval, ovoid or some similar shape to be used depending on the hernia or prolapse to be treated.

In this regard, while the surgical implant of the present disclosure may be useful for the repair of uterovaginal prolapse, it may also be used in a variety of surgical procedures including the repair of hernias.

To further reduce edge problems, the mesh of the present disclosure may have a circumferential member which extends, in use, along at least part of the perimeter of the implant to provide a substantially smooth edge as discussed below. In other words, the mesh may have at least one circumferential member (i.e., fiber, strand or the like) that extends around at least part of its circumference so that at least part of the perimeter of the implant is defined by the circumferential member. Alternatively, at least a part of the perimeter of the implant may be defined by more than one circumferential member, at the edge of the mesh.

The edge of the mesh, and hence the perimeter of the implant, can therefore be generally smooth and thus has significant advantages over conventional surgical meshes. Specifically, an implant having a smooth edge is less likely to cause edge extrusion or erosion.

Any amount of the perimeter of the implant may be defined by the circumferential member(s). In one embodiment, at least 50% of the perimeter of the implant may be defined by the circumferential member(s). In another embodiment, at least 80% of the perimeter of the implant may be defined by the circumferential member(s). In order to maximize the benefits of the mesh of the disclosure, it may be desirable in some embodiments to have 100% of the perimeter of the implant defined by the circumferential member(s). Thus, from about 50% to about 100% of the perimeter of the implant may be defined by the circumferential member(s), in embodiments from about 65% to about 95% of the perimeter of the implant may be defined by the circumferential member(s). The majority or whole of the perimeter of the mesh being smooth minimizes the risk of a rough edge causing edge erosion or infection.

The circumferential member(s) may be arranged in a variety of ways to provide the smooth edge or perimeter of the mesh of the present disclosure. In some cases it may be desirable to minimize the number of members utilized to form the perimeter. This simplifies the construction of the mesh, which is desirable not only for manufacture, but also because simpler structures are less likely to have defects which might be problematic after implantation. In embodiments, the perimeter of the mesh may be defined by one circumferential member.

In another embodiment, the mesh may have a plurality of circumferential members arranged at different radial locations. In order to provide an implant of a desired dimension, the periphery of the mesh outward of the desired circumferential member may be cut away such that one or more selected circumferential members form the perimeter of the implant as desired.

The circumferential members may also be arranged concentrically. A concentric arrangement of a plurality of circumferential members conveniently allows maintenance of the shape of the implant for different sizes of implant and provides the mesh with an even structure.

The circumferential members can also be arranged to join with one another in order to form an integral mesh. Alternatively, the mesh may additionally comprise transverse members which extend across the circumferential members joining the circumferential members.

The transverse members may extend radially from a central point to the perimeter of the implant. The transverse members may be arranged to provide substantially even structural strength and rigidity to the implant.

In some embodiments, it may be desirable to secure the mesh in place once it has been suitably located in the patient. The mesh can be secured in any manner known to those skilled in the art. Some examples include suturing the mesh to strong lateral tissue, gluing the mesh in place using a biocompatible glue, or using a surgical fastener, e.g., a tack, to hold the mesh securely in place.

In embodiments it may be advantageous to use a biocompatible glue since it is fairly quick to apply glue to the area around the surgical implant. Additionally, the mesh may include at least one capsule containing a biocompatible glue for securing the implant in place. In certain situations the mesh may include up to about four capsules containing a biocompatible glue which may be provided around the perimeter of the surgical implant. The capsules may be hollow thin-walled spheres from about 3 mm to about 5 mm in diameter and may be made of gelatin.

Any biocompatible glue within the purview of one skilled in the art may be used. In embodiments useful glues include fibrin glues and cyanoacrylate glues.

In another embodiment, the mesh of the present disclosure may be secured to tissue using a surgical fastener such as a surgical tack. Other surgical fasteners which may be used are within the purview of one skilled in the art, including staples, clips, helical fasteners, and the like.

In embodiments, it may be advantageous to use surgical tacks as a surgical fastener to secure the mesh. Tacks are known to resist larger removal forces compared with other fasteners. In addition, tacks only create one puncture as compared to the multiple punctures created by staples. Tacks can also be used from only one side of the repair site, unlike staples, clips or other fasteners which require access to both sides of the repair site. This may be especially useful in the repair of a vaginal prolapse, where accessing the prolapse is difficult enough without having to access both sides of the prolapse. Suitable tacks which may be utilized to secure the mesh of the present disclosure to tissue include, but are not limited to, the tacks described in U.S. Patent Application Publication No. 2004/0204723, the entire disclosure of which is incorporated by reference herein.

Suitable structures for other fasteners which may be utilized in conjunction with the mesh of the present disclosure to secure same to tissue are known in the art and can include, for example, the suture anchor disclosed in U.S. Pat. No. 5,964,783 to Grafton et al., the entire disclosure of which is incorporated by reference herein. Additional fasteners which may be utilized and tools for their insertion include the helical fasteners disclosed in U.S. Pat. No. 6,562,051 and the screw fasteners disclosed in International Patent Application No. PCT US04/18702, filed on Jun. 14, 2004, the entire disclosure of each of which are incorporated by reference herein.

The surgical fasteners useful with the mesh herein may be made from bioabsorbable materials, non-bioabsorbable materials, and combinations thereof. Suitable materials which may be utilized include those described in U.S. Patent Application Publication No. 2004/0204723 and International Patent Application No. PCT US04/18702, the entire disclosure of each of which are incorporated by reference herein. Examples of absorbable materials which may be utilized include trimethylene carbonate, caprolactone, dioxanone, glycolic acid, lactic acid, glycolide, lactide, homopolymers thereof, copolymers thereof, and combinations thereof. Examples of non-absorbable materials which may be utilized include stainless steel, titanium, nickel, chrome alloys, and other biocompatible implantable metals. In embodiments, a shape memory alloy may be utilized as a fastener. Suitable shape memory materials include nitinol.

Surgical fasteners utilized with the mesh of the present disclosure may be made into any size or shape to enhance their use depending on the size, shape and type of tissue located at the repair site.

The surgical fasteners, e.g., tacks, may be used alone or in combination with other fastening methods described herein to secure the mesh to the hernia, prolapse, or other repair site. For example, the mesh may be tacked and glued, or sutured and tacked, into place.

The surgical fasteners may be attached to the mesh in various ways. In embodiments, the ends of the mesh may be directly attached to the fastener(s). In other embodiments, the mesh may be curled around the fastener(s) prior to implantation. In yet another embodiment, the fastener may be placed inside the outer edge of the mesh and implanted in a manner which pinches the mesh up against the fastener and into the site of the injury.

According to another aspect of the present disclosure, there is provided a minimally invasive method of treating uterovaginal prolapse which includes the following steps: making an incision in the vaginal wall close to the opening of the vaginal cavity; making a subcutaneous cut, through the incision, over and surrounding the area of the prolapse, which cut is substantially parallel to the vaginal wall; and inserting a mesh according to the present disclosure through the incision, into the space defined by the cut.

Thus, a mesh according to the present disclosure can be inserted through a small incision (e.g., from about 1 cm to about 2 cm in length) in the region of the periphery or opening of the vaginal cavity. An incision in this position is easier for a surgeon to access than an incision deeper in the vaginal cavity. It is also more convenient to treat a vaginal prolapse by implanting a mesh of the present disclosure through such an incision.

In one embodiment, the incision may be at the anterior or posterior extremity of the prolapse sac of the vaginal cavity. This may be desirable, as prolapse most often occurs in the anterior or posterior vaginal wall, so positioning the incision in such a location allows the most convenient access to these parts of the vaginal wall.

Suitable placement of the mesh by minimally invasive techniques, particularly in the treatment of uterovaginal prolapse, requires the mesh to be as flexible as possible. Therefore the bioactive coating on the mesh should be strategically placed to ensure the mesh remains foldable, rollable, flexible, etc. In some embodiments, a flexible, less bulky mesh may be more easily handled in the repair of a prolapse by certain tools. Tools that may be used to carry out this procedure are known to those skilled in the art. An example of a suitable tool is disclosed in PCT Application No. PCT/GB02/01234, the entire disclosure of which is incorporated by reference herein. Any tool capable of properly inserting the mesh may ultimately be used.

Embodiments of the present disclosure will now be described, by way of example only, with reference to the accompanying drawings.

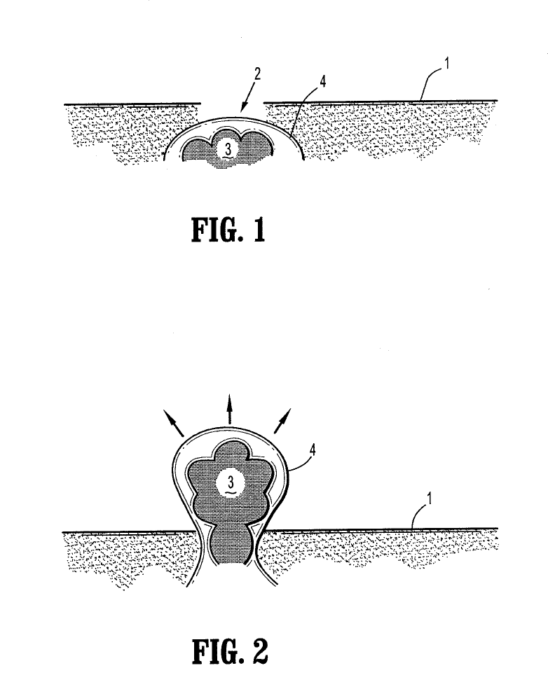

Referring to FIGS. 1 and 2, a hernia, vaginal prolapse or similar injury occurs when a fascial wall 1 ruptures, forming a defect 2, i.e. a weakening or, in this case, parting of the fascial wall 1. An organ 3, contained by the fascial wall 1 is then able to protrude through the defect 2. Such protrusion is illustrated in FIG. 2 and occurs particularly when pressure within the cavity defined by the fascial wall 1 is raised. For example, in the case of an inguinal hernia, when a patient coughs, intra-abdominal pressure is raised and the intestines may be pushed through the defect 2 in the abdominal wall.

While the organ 3 that may protrude through the defect 2 is usually still contained by some other membrane 4, the hernia, prolapse or such like is inevitably painful and liable to infection or other complications. An effective and desirable treatment is therefore to close the defect 2 and contain the organ 3 in its normal position.

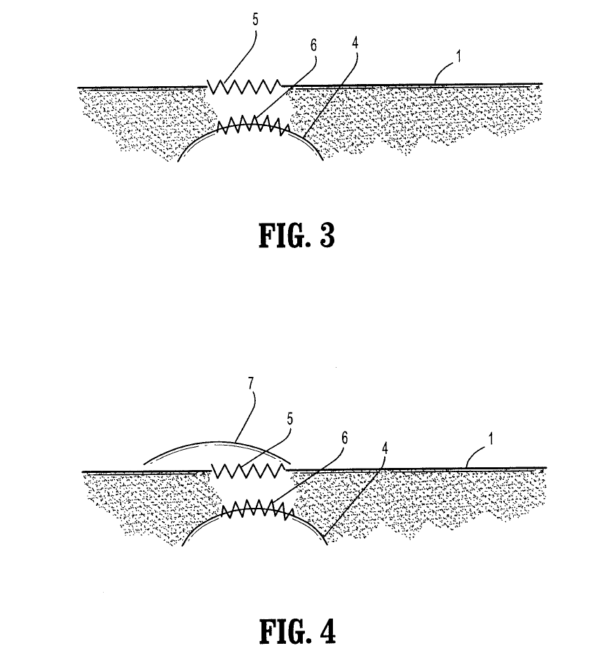

Referring to FIG. 3, a hernia or vaginal prolapse may be conventionally repaired by providing sutures 5 across the defect 2 to join the tissues of the fascial wall 1. In addition, it may be necessary to plicate (i.e., fold or reduce) the other membrane 4 as this may have stretched due to distension of the organ 3. Plication of the other membrane 4 corrects the stretching and helps to relieve pressure on the area of defect 2 during healing as the other membrane 4 can act to contain the organ 3 to some extent. Plication is generally achieved by applying sutures 6 to the other membrane 4.

Referring to FIG. 4, it is also a known method of treating hernias to provide, additionally or alternatively to sutures, a mesh 7 across defect 2. This allows for the defect 2 to be repaired without the parted tissues of the fascial wall 1 necessarily being brought together and for the defect to heal without the fascial wall 1 being pinched or tensioned to correct the defect 2.

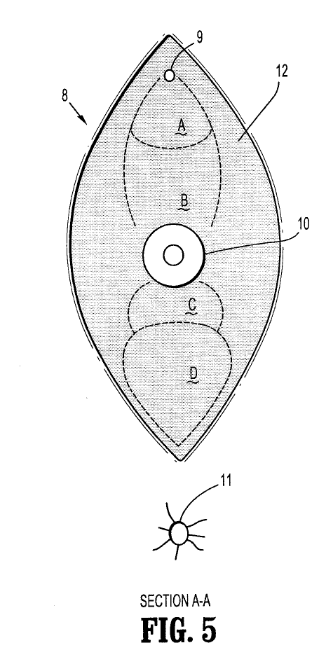

FIG. 5 schematically illustrates (a sagittal view of) the female human vaginal area. The vagina 8 is illustrated with its anterior portion (front) at the top of the diagram and the posterior portion (rear) at the bottom of the diagram. The opening of the urethra, or urethral meatus, 9 is at the forward or anterior end of the vagina 8. The central portion of the vagina 8 forms the vaginal cavity which terminates at the cervix 10. Spaced from the rearward or posterior end of the vagina 8 is the anus 11. Four areas A to D of the vaginal wall 12 are outlined in FIG. 5. These areas A to D are those areas of the vaginal wall 12 in which vaginal prolapse often occurs.

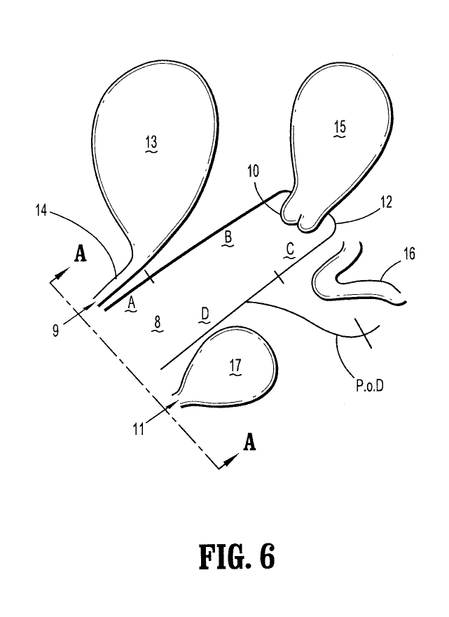

Referring to FIG. 6, which is a cross sectional view along the line A-A in FIG. 5, it can be more clearly seen that the wall 12 of the vagina 8 is bounded by the bladder 13 and urethra 14, the uterus 15, the small bowel 16 and rectum 17. The small bowel 16 and rectum 17 are separated by the Pouch of Douglas.

Area A is the lower one third of the anterior vaginal wall 12 (i.e. the one third nearest the entrance to the vaginal cavity) adjacent the bladder 13 and urethra 14. Prolapse in this area is referred to as anterior or, more specifically, urethracoele prolapse. Area B is the upper two thirds of the anterior vaginal wall 12. Prolapse in this area is referred to as anterior or, more specifically, cystocoele prolapse. The central area of the vaginal wall 12 in which the cervix 10 is located is adjacent the uterus 15 and prolapse in this area is referred to as central, uterine or vault prolapse. Area C is the upper one third of the posterior vaginal wall 12. This area of the vaginal wall 12 is adjacent the small bowel 16 and prolapse in this area is referred to as posterior or enterocoele prolapse. Finally, area D is the lower two thirds of the posterior vaginal wall and is adjacent the rectum 17. Prolapse in this area is generally referred to as posterior or rectocoele prolapse.

Conventionally, any of the above types of hernia have been treated by providing sutures in the area of the prolapse. For example, the extent of the defect causing the prolapse is first identified by the surgeon. Lateral sutures, i.e. sutures from one side to the other of the vaginal wall 12 as seen in FIG. 5, or right to left rather than anterior to posterior, are provided across the area of the defect. This joins the parted tissues of the vaginal wall and repairs the defect. The organ protruding through the vaginal wall is therefore contained. Disadvantages of this technique include anatomical distortion of the vagina due to tensioning of the wall by the sutures to repair the defect.

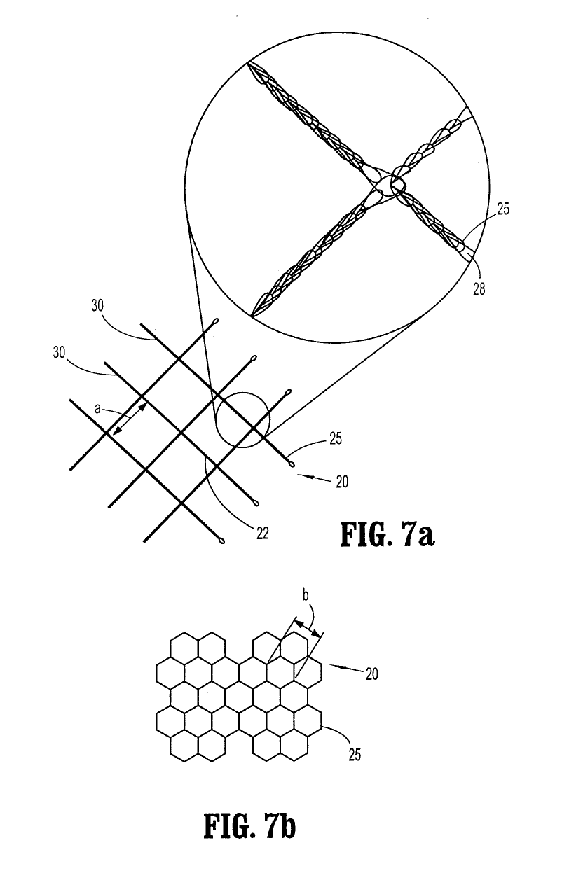

Turning FIGS. 7A and 7B, a surgical implant for use in the repair of vaginal prolapse in accordance with an embodiment of the present disclosure comprises a coated mesh 20. The mesh is comprised of strands 22. The strands may be less than about 600 μm, and approximately from about 150 μm to about 600 μm in diameter. The strands are arranged such that they form a regular network and are spaced apart from each other such that, for a diamond shaped mesh, a space of from about 2 mm to about 5 mm exists between the points where the strands of the mesh interact with each other as depicted in FIG. 7A. In a hexagonal net arrangement, the space is from about 2 mm to about 5 mm between opposite diagonal points where the strands of the mesh interact as depicted in FIG. 7B. In yet other embodiments, it is envisioned that pore sizes greater than 0.5 mm are desirable for tissue ingrowth.

It may be desirable to space the strands as far as part as possible to allow blood to pass through the implant and reduce the mass of the implant, while providing the mesh with sufficient tensile strength and elasticity to be effective. It can therefore be appreciated that considerable variability in the maximum spacing between the strands can be achieved depending on the material from with the strands are made and the net pattern in which the strands are arranged.

In the embodiment shown in FIG. 7A, the strands are arranged in a diamond net pattern, however any pattern which provides suitable tensile strength and elasticity may be used. For example a hexagonal net pattern may be used as shown in FIG. 7B. Ideally, in order to reduce the overall mass of the implant, the strands 22 should have as narrow a diameter as possible while still providing the mesh 20 with suitable tensile strength and elasticity.

The strands 22 of the mesh 20 may be comprised of at least two filaments 25 arranged to interact such that pores 28 are formed between the filaments 25. The pores 28 formed between the filaments 25 may be from about 50 μm to about 200 μm in the diameter, which permits fibroblast through-growth to occur. This fibroblast through-growth secures the implant 20 in place within the body. The suitably sized pores allow the implant 20 to act as a scaffold to encourage the lay down of new tissue. The lay down of new tissue promotes the healing of the hernia or proplapse being treated.

The filaments 25 may be formed from any biocompatible material. In one embodiment the filaments 25 may be formed from polyester, wherein each polyester filament 25 is about 0.09 mm in diameter. In the embodiment shown the filaments 25 of the strands 22 are knitted together using a warp knit to reduce the possibility of fraying of the filaments 25 and strands 22.

The fine warp knit of the filaments 25 provide a surgical implant which is flexible in handing and which can be easily cut into different shapes and dimensions. As the strands 22 are formed using warp knit, the possibility of fraying of the edge of the surgical implant 20 following production or cutting of the surgical implant 20 is reduced.

Other methods of reducing fraying of the filaments are heat treatment, laser treatment or the like, to seal the edges of the surgical implant.

The mesh 20 may be supplied in any shape or size and cut to the appropriate dimensions as required by the surgeon.

It can be appreciated that cutting of the mesh will produce an unfinished edge. Due to the sparse nature of the strands that form the mesh and their narrow diameter, this unfinished edge does not suffer from the same problems as edges of meshes of the prior art as previously described. In other words, the edge produced is not rough and jagged such that it increases the likelihood of extrusion of the edge of the mesh in situ or the chance of infection.

As discussed above, an advantage of the mesh of the present disclosure is that it allows the production of a mesh suitable for use in hernia repair which allows substantially less foreign material to be left in the body.

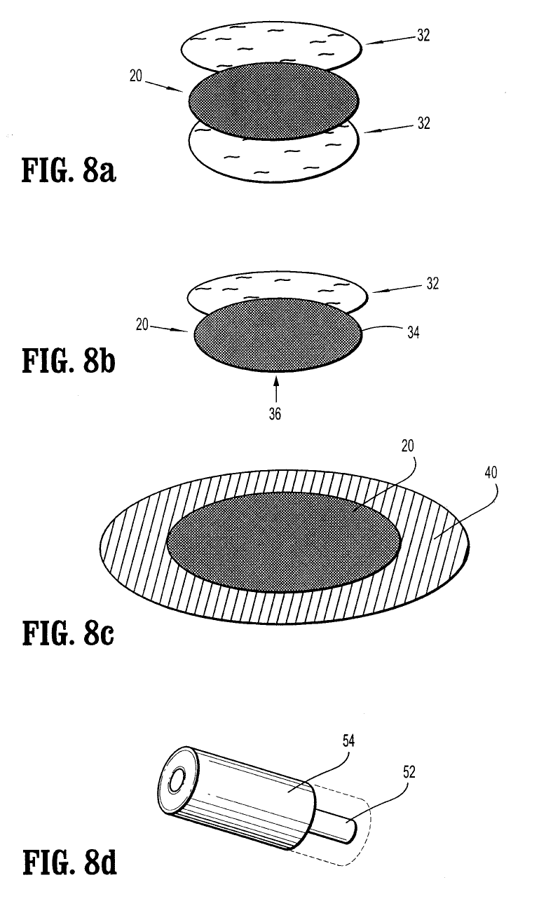

Referring to FIGS. 8A and 8B, the mesh includes a bioactive coating 32. The bioactive coating 32 may, in some embodiments, comprise a layer of absorbable material possessing at least one bioactive agent, wherein the coating layer has a thickness greater than that of the strands 22 of the mesh 20. For example, the thickness of the layer of coating material may be about 1 mm to about 2 mm. The strands of the mesh 20 may be entirely embedded in the bioactive coating 32 such that the outer surface of the mesh 20 is covered entirely by the bioactive coating 32. In effect, the entire surgical implant may be encased in the bioactive coating as shown in FIG. 8A.

Thus, the surgical implant has no gaps or holes on its surface. This has the advantage of reducing the likelihood of bacteria becoming lodged on the strands of the mesh 20 before implantation of the mesh 20. Furthermore, the bioactive coating 32 makes the mesh 20 more substantial and less flexible such that it is more easily handled by a surgeon. This is particularly useful when it is desired to place the mesh in a desired location in a conventional, open surgical procedure.

In an alternate embodiment shown in FIG. 8B, the bioactive coating 32 comprises a layer of coating material applied to one face 34 of the mesh 20, such that the mesh has a first face 34 on which the coating material has been applied and a second face 36 on which the coating material has not been applied. Thus, the first and second faces 34 and 36 each have different characteristics.

In another embodiment depicted in FIG. 8C, a surgical implant may be desired utilizing the releasable attachment of the mesh 20 to a backing strip 40. The backing strip may be formed from a plastic material and may be adhered to the surgical implant using a releasable adhesive. The backing strip 40 causes the mesh 20 to be more substantial and less flexible such that it is more easily handled by a surgeon. Following the suitable placement of the mesh 20, the backing strip 40 can be removed from the mesh 20, the mesh 20 being retained in the body and the backing material 40 being removed by the surgeon. An implant possessing backing strip 40 applied to mesh 20 means the mesh 20 benefits from reduced mass but the mesh 20 and backing strip 40 together may provide desirable characteristics for surgical handling.

As shown in FIG. 8D, in a further embodiment the filaments of the mesh may be comprised from bicomponent microfibers. The bicomponent microfibers may include a core 52 (cutaway section shows core region) and surface material 54. The surface material 54 is designed such that it is absorbed by the body in a matter of hours, while the core material 52 remains in the body for a longer period to enable tissue in growth.

Suitable bicomponent microfibers include a polypropylene non-absorbable portion and a polylactic acid absorbable portion. The surface material 54 is present during the surgical procedure when the mesh is being inserted and located in the patient, and provides the mesh with characteristics desirable for surgical handling. Following a period of insertion in the body, typically a few hours, the surface material 54 is absorbed into the body leaving only the core material 52 of the filaments in the body.

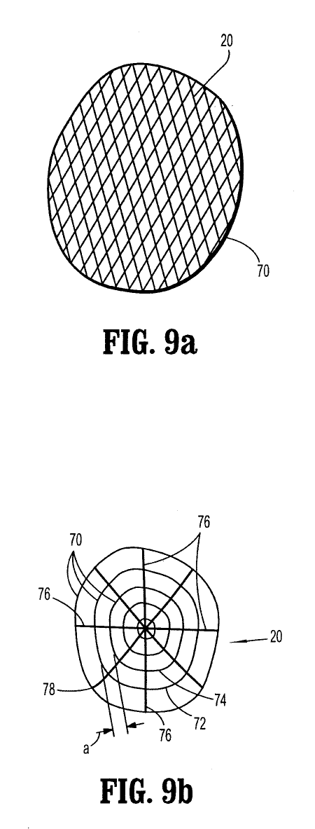

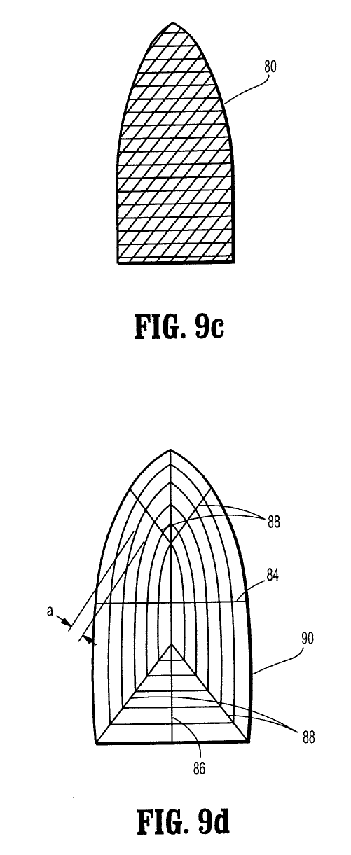

Referring to FIGS. 9A and 9B, a further embodiment of the mesh may include perimeter strands. Typically the mesh 20 is circular or the like in shape and the perimeter strand can be generally referred to as a circumferential strand 70.

In the example shown in FIG. 9A, one strand 70 runs around the circumference of the oval shape of the mesh 20. In another embodiment, several circumferential strands may be present, each circumferential strand extending over one side of the oval mesh, e.g., around half the circumference of the mesh, a quarter of the circumference of the mesh, etc.

As shown in FIG. 9B, the circumferential strands 70 may also be arranged concentrically and each extend around the mesh 20 at a different radial location. An outer circumferential strand 78 extends around the perimeter of the mesh 20, and further circumferential strands 72 and 74 are arranged inwardly of the outer circumferential strand forming a perimeter spaced by a distance (a). The distance (a) between adjacent circumferential members 78, 72 and 74 can vary and, in this example, is about 20 mm.

As also depicted in FIG. 9B, transverse strands 76 may be present which extend from the center of the oval mesh to points on the perimeter of the mesh 78. In this example, four transverse strands 76 are provided across the diameter of the mesh 20, dividing the mesh into eight angularly equal portions.

The mesh 20 of this embodiment may be formed from materials as previously described. Depending on the material chosen, the mesh may be woven, knitted or extruded as one piece, or individual or groups of strands can be extruded separately and joined to one another.

Such a construction as described above provides a mesh 20 with sufficient tensile strength to repair defects causing vaginal prolapse while having minimal bulk. Similarly, such a construction provides a flexible yet resilient mesh for handling.

Referring to FIGS. 9C and 9D, meshes 80 and 90 may be produced having angled sides. These meshes have a similar structure to that described with reference to FIGS. 9A and 9B. Further, the mesh may have transverse members arranged only to extend towards the perimeter of the mesh, rather than all being across the diameter of the mesh. This provides a more uniform structure. More specifically, referring to FIG. 9D, the mesh may have a transverse member 84 extending along its axis of symmetry, a transverse member 86 bisecting the axis of symmetry, and four further transverse members 88 extending from the axis of symmetry to the perimeter of the mesh 90.

In addition to the pores provided by the combination of filaments which form the strands of the mesh, pores can be provided by rings of polypropylene positioned at the intersection of the circumferential and transverse members.

Alternatively, pores may be formed by the spacing of the transverse members, such that pores of a size of from about 50 μm to about 200 μm suitable for enabling tissue ingrowth exist between the transverse members.

To secure the mesh to a suitable location in the body, a number of methods can be used. The bioactive coating may be tacky and thus suitable to hold the mesh in place until it is secured by tissue ingrowth.

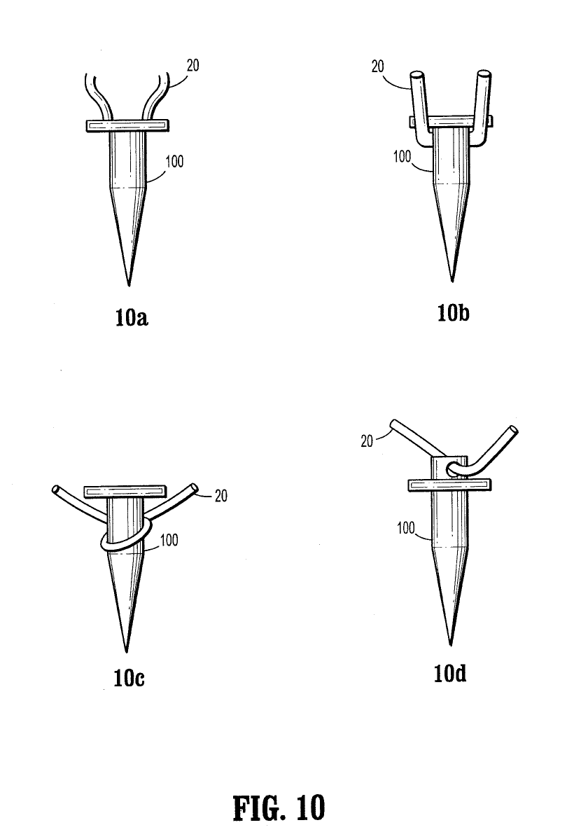

Alternatively, the surgical implant can utilize fasteners such as tacks to secure the mesh in place. Referring to FIGS. 10A-D, a variety of different tacks 100 can be used to secure the mesh 20 into place. The mesh 20 can be directly attached to the tack as seen in FIGS. 10A and 10D. Alternatively, the mesh 20 can be placed underneath the head of the tack prior to implantation as demonstrated by FIGS. 10B and 10C. Also, the edges of the mesh can be wrapped around a tack to further secure the two devices.

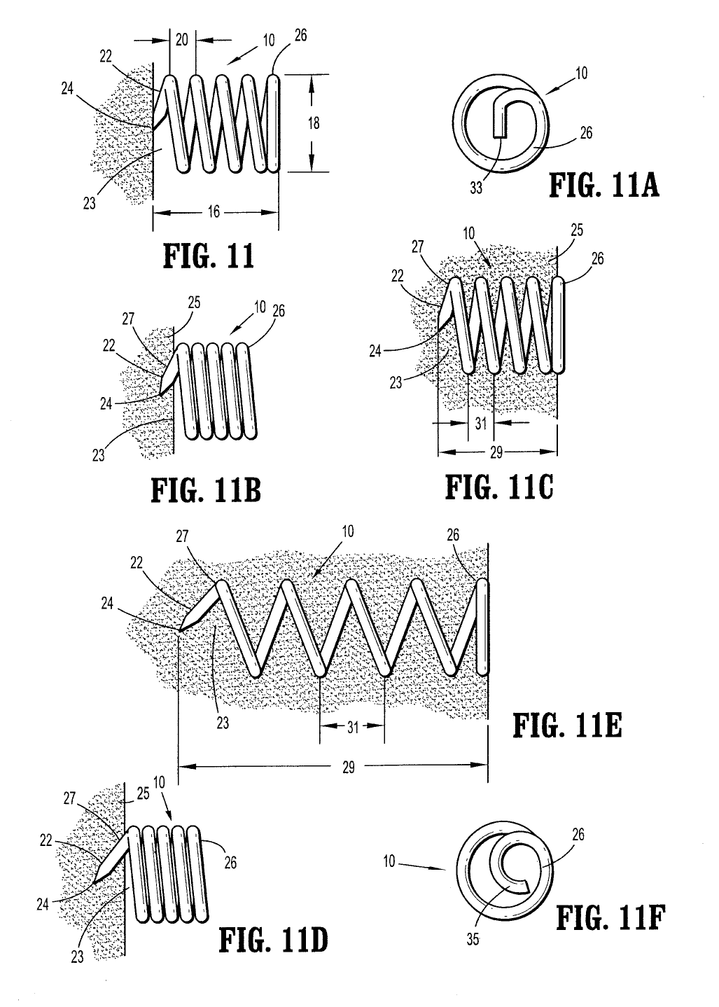

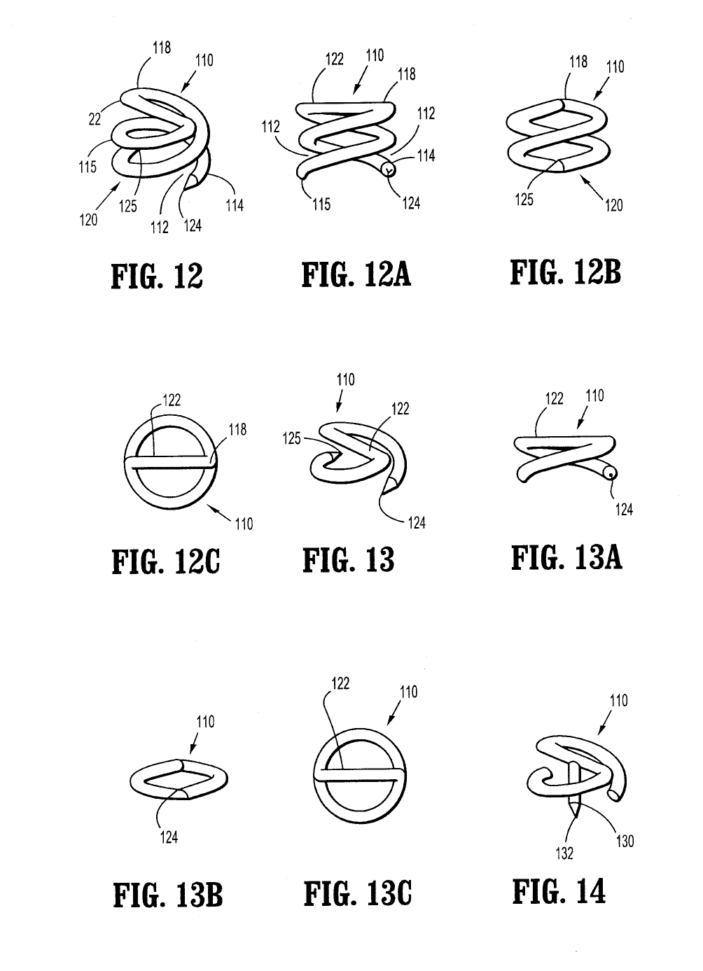

Configurations of additional fasteners which may be utilized to attach a mesh of the present disclosure to tissue are helical fasteners depicted in FIG. 11 (including FIGS. 11A-F), FIG. 12 (including FIGS. 12A-C), FIG. 13 (including FIGS. 13A-C), and FIG. 14. The helical fasteners of FIGS. 11-14 correspond to FIGS. 1-4 of U.S. Pat. No. 6,562,051, the entire disclosure of which is incorporated by reference herein.

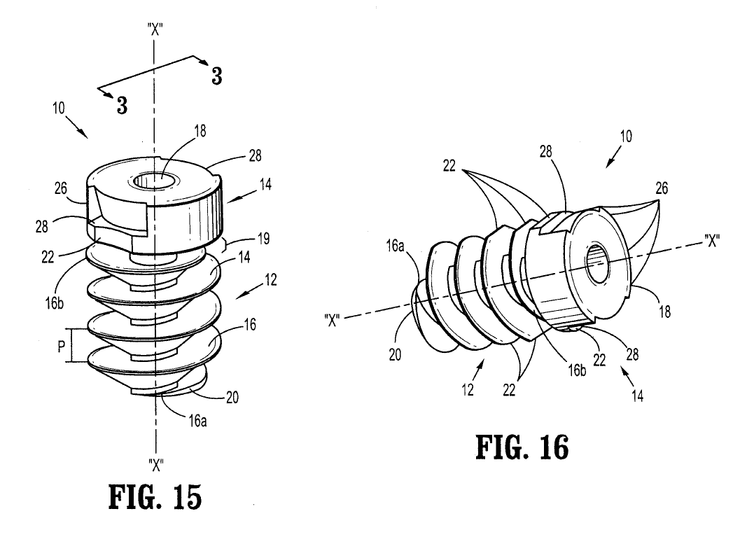

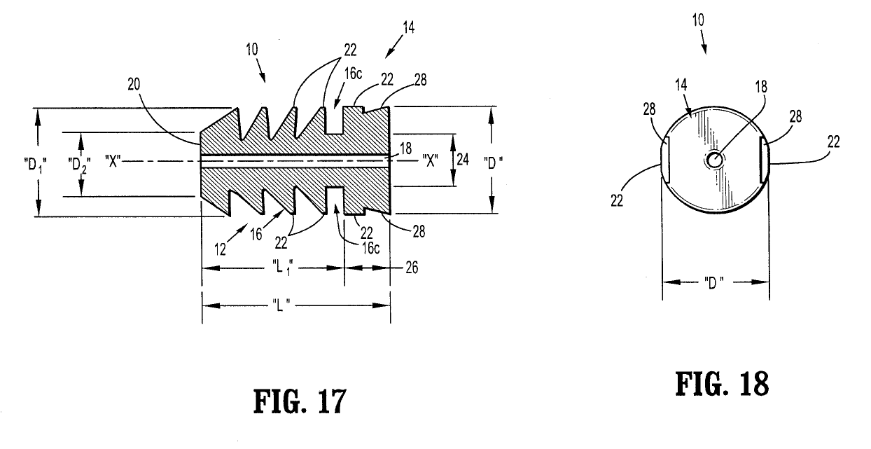

Other fasteners which may be utilized to attach a mesh of the present disclosure to tissue are the screw fasteners depicted in FIGS. 15, 16, 17 and 18. The screw fasteners of FIGS. 15-18 correspond to FIGS. 1-4 of International Patent Application PCT US04/18702, filed on Jun. 14, 2004, the entire disclosure of which is incorporated by reference herein.

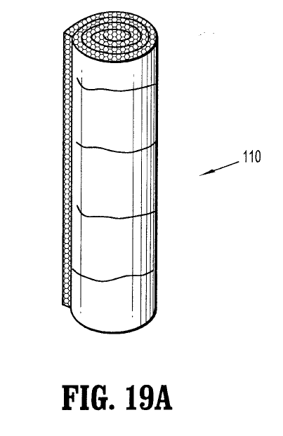

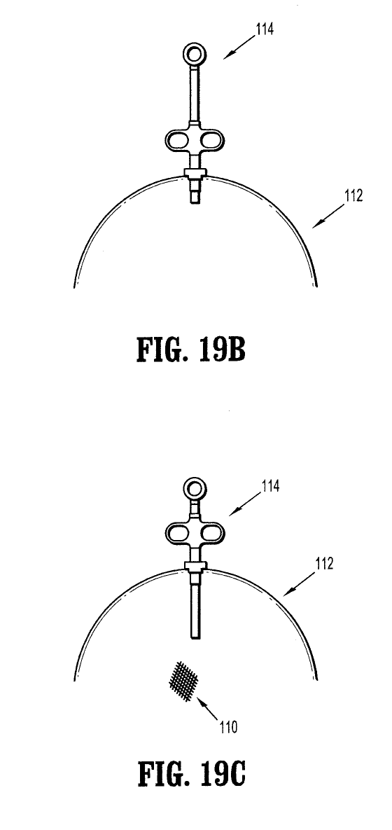

In use, the bicomponent microfiber of FIG. 8D may be formed into a surgical mesh of the disclosure and transported laparoscopically into a body cavity. As depicted in FIG. 19A the bicomponent microfiber surgical mesh 110 may be rolled into a shape suitable for transport in a laparoscopic deployment device. FIG. 19B is an illustration of the laparoscopic deployment device 114, which may be inserted into the body cavity 112. As illustrated in FIG. 19C, mesh 110 is transferred into the body cavity. Due to the rigidity of the absorbable outer layer, the mesh will return to its original geometric shape. The surgical mesh may then be attached to the area of tissue in need thereof.

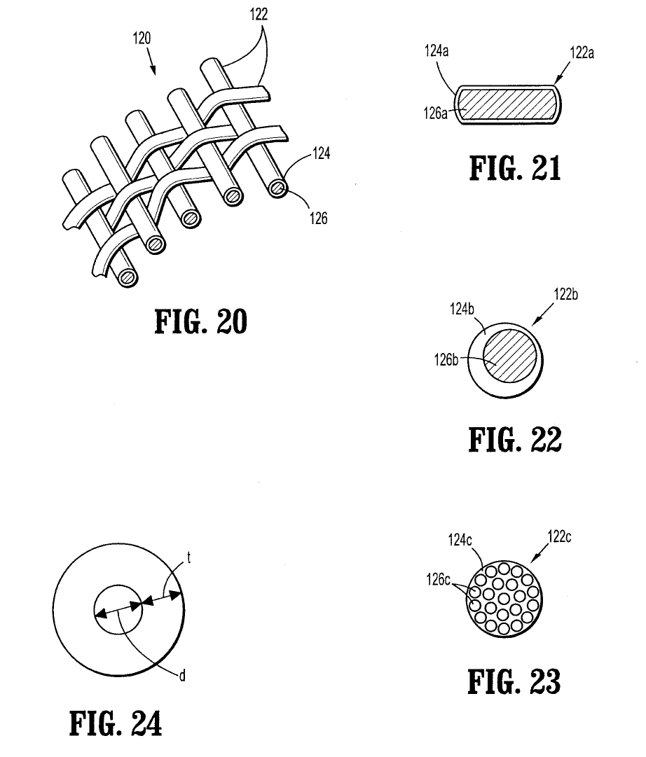

Alternatively, FIG. 20 illustrates an embodiment of a surgical mesh 120 assembled from a plurality of bicomponent fibers 122 in accordance with the present disclosure. The bicomponent fibers 122 are woven to form an intertwining structure or pattern of fibers. The bicomponent fibers 122 are monofilament fibers which are co-extruded from two distinct polymers to exhibit a concentric sheath-core arrangement. Fiber 122 has a round cross-sectional shape including a core 126 surrounded by a sheath 124, the core 126 and sheath 124 both having a common center. Other cross-sectional shape are envisioned, such as a flat cross-sectional shape as illustrated by fiber 122a in FIG. 21, as well as other modified cross-sections which may be co-extruded to generate fibers with more complex profiles as envisioned by those skilled in the art. As illustrated in FIG. 22, bi-component fibers may also exhibit an eccentric sheath-core arrangement. Fiber 122b includes an off-center core 126b surrounded by sheath 124b.

Bicomponent fibers may also be multifilament fibers which are spun and processed from microdenier filaments of core material. FIG. 23 illustrates a fiber 122c exhibiting an islands-in-the-sea arrangement where two or more “island,” or core polymer filaments 126c are surrounded by a bioabsorbable “sea,” or sheath polymer 124c. This arrangement may provide for very fine strands of island polymer filaments 126c to be effectively handled by manufacturing equipment to spin and form fiber 122c. Core polymer filaments 126c may be arranged so as to be generally non-intersecting along their length. Although not necessarily parallel, core polymer filaments 126c may be generally free from entanglement or interlacing over a substantial portion of their length. Alternatively, core polymer filaments 126c may be woven, braided, or entangled by various processes within the purview of those skilled in the art. Once the core polymer filaments 126c are in place, the bioabsorbable sheath polymer 124c is dissolved away leaving the core polymer filaments 126c in place. In embodiments, as little as about three and as many as about fifty or more core polymer filaments 126c may be handled effectively to form a single fiber 122c.

FIG. 24 illustrates a biocomponent fiber of the present disclosure having a core with diameter “d” and a sheath with thickness “t”.

It should be understood that various combinations of bicomponents fibers and/or microfibers may be used to fabricate the mesh according to the present disclosure. For example, the mesh may have outer edges that include fibers having a stiffer sheath (i.e., having a higher modulus of elasticity) than the fibers that make up the inside of the mesh to facilitate smooth manipulation of the mesh during implantation, e.g., ease in rolling up the mesh for insertion through a cannula. The mesh may also have outer edges that include fibers having a slower degrading sheath polymer or a larger cross-section of sheath polymer than the fibers making up the inside of the mesh thereby providing additional bulk during suturing or stapling of the mesh to the tissue wall.

While the surgical meshes are especially suitable for surgical repair of hernias, it is envisioned that the meshes can be used in connection with other surgical procedures requiring repair of soft tissue defects, such as muscle or wall tissue defects, pelvic organ prolapse, and urinary incontinence, for example. The meshes of the present disclosure can be in the from of sheets, patches, slings, suspenders, and other implants and composite materials, such as pledgets, buttresses, wound dressings, drug delivery devices, and the like.

While several embodiments of the disclosure have been described, it is not intended that the disclosure be limited thereto, as it is intended that the disclosure be as broad in scope as the art will allow and that the specification be read likewise. Therefore, the above description should not be construed as limiting, but merely as exemplifications of embodiments of the present disclosure. Various modifications and variations of the sheath and core polymers of the bicomponent fibers and microfibers of the mesh, as well as methods of assembling the bicomponent fibers and microfibers into a mesh, will be apparent to those skilled in the art from the foregoing detailed description. Such modifications and variations are intended to come within the scope and spirit of the claims appended hereto.

What is claimed is:

1. A surgical mesh comprising at least one bicomponent fiber, the bicomponent fiber comprising:
a non-absorbable inner core comprising a first polymer; and
a sheath surrounding at least a portion of the core comprising an absorbable rigid surface material comprising a second polymer,
wherein the surgical mesh is more stiff prior to implantation and less stiff following implantation in tissue.

2. The surgical mesh according to claim 1, wherein the first polymer is less stiff compared to the second polymer.

3. The surgical mesh according to claim 1, wherein the second polymer has an increased stiffness compared to the first polymer.

4. The surgical mesh according to claim 1, wherein upon degradation of the second polymer, stiffness of the surgical mesh decreases.

5. The surgical mesh according to claim 1, wherein the second polymer has a glass transition temperature which is higher than 25° C.

6. The surgical mesh according to claim 1, wherein the second polymer has a glass transition temperature which is less than 37° C.

7. The surgical mesh according to claim 1, wherein the first polymer is selected from the group consisting of polyesters, polyolefins, polyamides, fluoropolymers, and combinations thereof.

8. The surgical mesh according to claim 1, wherein the second polymer is selected from the group consisting of poly(lactide), poly(glycolide), p-dioxane, poly(trimethylene carbonate), poly (e-caprolactone), poly(orthoester), and combinations thereof.

9. The surgical mesh according to claim 1, wherein the absorbable rigid surface material absorbs within a period of from about 2 days to about 180 days.

10. The surgical mesh according to claim 1, wherein the non-absorbable inner core is present in an amount of from about 50 to about 80 percent by weight of the bicomponent fiber.

11. The surgical mesh according to claim 1, wherein the absorbable rigid surface material is present in an amount of from about 20 to about 50 percent by weight of the bicomponent fiber.

12. The surgical mesh according to claim 1, wherein the surgical mesh comprises a hernia mesh.

13. The surgical mesh according to claim 1, further comprising a bioactive agent selected from the group consisting of antimicrobials, antibacterials, anti-fungals, antibiotics, anti-viral agents, analgesics, antiadhesives, anesthetics, anti-inflammatories, antispasmodics, hormones, growth factors, muscle relaxants, antineoplastics, immunogenic agents, immunosuppressants, steroids, lipids, narcotics, lipopolysaccharides, polysaccharides, polypeptides, proteins, enzymes, and combinations thereof.
